(19) United States
(12) Patent Application Publication
(10) Pub. No.: US 2025/0290014 A1
(43) Pub. Date: Sep. 18, 2025

(54) DETERGENT COMPOSITION

(71) Applicant: Saraya Co., Ltd., Osaka-shi, Osaka (JP)

(72) Inventors: Akiko SHIMADA, Kashiwara (JP); Michiaki ARAKI, Kashiwara (JP); Abigail Loren Tung UY, Kashiwara (JP); Yoshihiko HIRATA, Kashiwara (JP)

(73) Assignee: Saraya Co., Ltd., Osaka-shi, Osaka (JP)

(21) Appl. No.: 18/860,519

(22) PCT Filed: Apr. 27, 2023
(86) PCT No.: PCT/JP2023/016695

§ 371 Date: Oct. 25, 2024

(30) Foreign Application Priority Data
Apr. 27, 2022 (JP) 2022-073656

(51) Int. Cl. C11D 10/04; C11D 3/20; C11D 9/00

(52) U.S. Cl. CPC C11D 10/04; C11D 3/2096; C11D 9/007

(57) ABSTRACT

This invention provides a cleaning agent composition in which thermal deterioration is suppressed. The cleaning agent composition is a liquid cleaning agent composition comprising (a) fatty acid salts and (b) a sophorose lipid, wherein the fatty acid salts (a) comprise (a-1) at least one member selected from the group consisting of salts of C₁₄-C₂₂ saturated fatty acids and (a-2) at least one member selected from the group consisting of salts of C₁₄-C₂₂ unsaturated fatty acids, wherein
A) the total amount of the component (a) is 2-35 mass % based on the cleaning agent composition taken as 100 mass %;
B) the proportion of the component (a-2) is 20 parts by mass or more based on 100 parts by mass of the total amount of the component (a); and
C) the proportion of the component (b) is 3 parts by mass or more per 100 parts by mass of the component (a-2).

### TECHNICAL FIELD

The present invention relates to a cleaning agent composition. More specifically, the present invention relates to a cleaning agent composition comprising a saturated fatty acid salt and an unsaturated fatty acid salt as fatty acid salts, wherein the weaknesses of each fatty acid salt are improved while utilizing their advantages. The present invention also relates to a method for improving weaknesses in a cleaning agent composition comprising an unsaturated fatty acid salt as a fatty acid salt.

### BACKGROUND ART

Cleaning agents are used for washing the body including hands, face, and head, and hair; dishes, houses, clothes, and anything in daily life. Cleaning agents have the effect of removing stains, removing bacteria to prevent infection, and/or removing and suppressing odor, as well as the effect of making the user feel refreshed by washing. Such cleaning agents are required to be stable against light and heat, to be less irritating to the skin, to ensure good lathering and good foam quality, and/or to impart an excellent feel during use (e.g., no unpleasant slippery feel during rinsing and/or no squeaky feel or skin tightening sensation after washing).

In particular, a slippery feel during rinsing and a squeaky feel and skin tightening sensation after washing are known to be caused by synthetic surfactants or fatty acid salts (soap base material), which are contained as cleaning and foaming components in cleaning agents, remaining on the skin. Several methods have been proposed to solve these problems. For example, it has been reported that problems such as a slippery feel, a squeaky feel, skin tightening sensation during rinsing or after washing can be solved by incorporating a sophorose lipid (which may be simply referred to below as an “SL”), which is a natural surfactant, into cleaning agents to inhibit synthetic surfactants or fatty acid salts from adhering to and remaining on the human body (see, for example, Patent Literature (PTL) 1).

SLs, which are a type of glycolipid biosurfactant produced by microorganisms (natural surfactant) (Non-patent Literature (NPL) 1), are less irritating to the skin, have high biodegradability, and are good for the natural environment. In this sense, SLs have properties in common with alkaline soap (fatty acid salt), which is a cleaning agent with a high level of safety and biodegradability. Thus, the present applicant previously found that adding an SL to a soap base material (fatty acid salt) improves weaknesses of soap, such as low solubility in water and poor foaming under hard water conditions, while utilizing the advantages of soap (high level of safety and biodegradability), and further improves moisture-retaining properties, and the present applicant proposed a cleaning agent composition comprising a soap base material (fatty acid salt) and an SL (see, for example, PTL 2). The present applicant also proposed a cleaning agent composition that comprises a soap base material (fatty acid salt) and an SL in combination to prevent clogging at the dispensing outlet when stored in a dispenser and used, while improving stability at low temperature (see PTL 3).

### CITATION LIST

### Patent Literature

PTL 1: JP2009-275145
PTL 2: JP2006-083238
PTL 3: WO2018/164204

### Non-Patent Literature

NPL 1: Canadian Journal of Chemistry, 39, 846 (1961)

### SUMMARY OF INVENTION

### Technical Problem

As mentioned above, by incorporating a fatty acid salt, which is a soap base material, and an SL into a clearing agent, weaknesses of soap, such as low solubility in water and poor foaming under hard water conditions, can be improved while utilizing the advantages of soap (high level of safety and biodegradability), and further, skin tightening sensation and a squeaky feel after washing can be suppressed.

However, according to the findings of the present inventors, alkali metal salts of C₁₂ saturated fatty acid (lauric acid), which are lauric acid soap in soap base materials, have the problem of being highly irritating to the skin. In contrast, myristic acid soap, palmitic acid soap, and stearic acid soap, etc., which are alkali metal salts of saturated fatty acids having 14 or more carbon atoms in soap base materials, are less irritating to the skin (see Experimental Example 1), but are problematic in that a lot of insoluble metallic soap is formed during rinsing, which leaves a strong squeaky feel or skin tightening sensation after washing and causes the skin to become dry. These problems with saturated fatty acid salts may be solved by adding a salt of an unsaturated fatty acid having an unsaturated bond, in addition to a saturated fatty acid salt. However, unsaturated fatty acids, especially linoleic acid, are easily deteriorated by heat, which causes coloring and odor generation over time.

A primary object of the present invention is to suppress the deterioration of an unsaturated fatty acid salt due to heat over time in a cleaning agent composition comprising a fatty acid salt, in particular an unsaturated fatty acid salt, and an SL. “Deterioration” as used herein refers to the phenomenon caused by decomposition of an unsaturated fatty acid salt due to heat, and specifically includes at least one of the following phenomena: formation of low-molecular-weight compounds, coloring, and odor generation, caused by decomposition of an unsaturated fatty acid.

Another object of the present invention is to reduce skin irritation during use in a cleaning agent composition comprising a fatty acid salt, in particular an unsaturated fatty acid salt, and an SL.

Another object of the present invention is to suppress the amount of metallic soap produced by hard water components in tap water during rinsing to improve a squeaky feel during rinsing and skin tightening sensation after rinsing in a cleaning agent composition comprising a fatty acid salt, in particular an unsaturated fatty acid salt, and an SL.

### Solution to Problem

The present inventors conducted extensive research and found that the above problems can be solved by preparing a cleaning agent composition comprising a specific saturated fatty acid salt and a specific unsaturated fatty acid salt as fatty acid salts, and an SL in specific proportions.

The present invention has been completed as a result of further research based on this finding and encompasses the following embodiments.

### (I) Cleaning Agent Composition

(I-1) A liquid cleaning agent composition comprising:
   (a) fatty acid salts; and
   (b) an SL,
   wherein
   the fatty acid salts (a) comprise (a-1) at least one member selected from the group consisting of salts of C₁₄-C₂₂ saturated fatty acids and (a-2) at least one member selected from the group consisting of salts of C₁₄-C₂₂ unsaturated fatty acids, and
   the components are contained in the following proportions:
A) the total amount of the component (a) is 2 to 35 mass % based on the cleaning agent composition taken as 100 mass %;
B) the proportion of the component (a-2) is 20 parts by mass or more based on 100 parts by mass of the total amount of the component (a); and
C) the proportion of the component (b) is 3 parts by mass or more per 100 parts by mass of the component (a-2).
(I-2) The cleaning agent composition according to (I-1), wherein B) the proportion of the component (a-2) is 20 to 95 parts by mass based on 100 parts by mass of the total amount of the component (a).
(I-3) The cleaning agent composition according to (I-1) or (I-2), wherein C) the proportion of the component (b) is 3 to 500 parts by mass per 100 parts by mass of the component (a-2).
(I-4) The cleaning agent composition according to any one of (I-1) to (I-3), wherein A) the total amount of the component (a) is 3 to 32 mass % based on the cleaning agent composition taken as 100 mass %.
(I-5) The cleaning agent composition according to any one of (I-1) to (I-4),
   wherein
   the component (a-1) is a salt of at least one member selected from the group consisting of myristic acid, palmitic acid, isopalmitic acid, stearic acid, isostearic acid, and arachidic acid, and
   the component (a-2) is a salt of at least one member selected from the group consisting of oleic acid, linoleic acid, linolenic acid, palmitoleic acid, arachidonic acid, eicosenoic acid, erucic acid, eicosadienoic acid, docosadienoic acid, and mead acid.

### (II) Method for Suppressing Thermal Deterioration of Cleaning Agent Composition

(II-1) A method for suppressing thermal deterioration of a cleaning agent composition comprising (a) fatty acid salts, the fatty acid salts (a) comprising (a-1) at least one member selected from the group consisting of salts of C₁₄-C₂₂ saturated fatty acids and (a-2) at least one member selected from the group consisting of salts of C₁₄-C₂₂ unsaturated fatty acids, the method comprising incorporating (b) an SL in an amount of 3 mass % or more per 100 parts by mass of the component (a-2) in the cleaning agent composition.
(II-2) The method according to (II-1), wherein the cleaning agent composition comprises at least a salt of at least one unsaturated fatty acid selected from the group consisting of linoleic acid, linolenic acid, eicosadienoic acid, docosadienoic acid, mead acid, and arachidonic acid as the component (a-2).
(II-3) The method according to (II-1) or (II-2), wherein the cleaning agent composition comprises the component (a) and the component (a-2) in the following proportions:
A) the total amount of the component (a) is 2 to 35 mass % based on the cleaning agent composition taken as 100 mass %; and
B) the proportion of the component (a-2) is 20 parts by mass or more based on 100 parts by mass of the total amount of the component (a).
(II-4) The method according to (II-3), wherein A) the total amount of the component (a) is 3 to 32 mass % based on the cleaning agent composition taken as 100 mass %.
(II-5) The method according to (II-3), wherein B) the proportion of the component (a-2) is 20 to 95 parts by mass based on 100 parts by mass of the total amount of the component (a).
(II-6) The method according to any one of (II-1) to (II-5), wherein the proportion of the component (b) is 3 to 500 parts by mass per 100 parts by mass of the component (a-2).

### Advantageous Effects of Invention

According to the present invention, a cleaning agent composition in which the deterioration of an unsaturated fatty acid salt due to heat is suppressed can be provided by containing a specific saturated fatty acid salt and a specific unsaturated fatty acid salt as fatty acid salts, and an SL in specific proportions. More specifically, the present invention can provide a cleaning agent composition with good thermal stability in which the formation of low-molecular-weight compounds, coloring, and/or odor generation caused by decomposition of, in particular, linoleic acid salts among unsaturated fatty acid salts is suppressed.

The present invention also provides a method for suppressing the deterioration due to heat in a cleaning agent composition containing, in particular, a linoleic acid salt as an unsaturated fatty acid salt. More specifically, the present invention provides a method for suppressing the formation of low-molecular-weight compounds, coloring, and/or odor generation in a cleaning agent composition containing an unsaturated fatty acid salt.

According to the present invention, a cleaning agent composition having at least one of the following effects in addition to the above effects can be provided by containing a specific saturated fatty acid salt and a specific unsaturated fatty acid salt as fatty acid salts, and an SL in specific proportions:
(i) suppression of metallic soap formation;
(ii) low skin irritation; and
(iii) good stability at low temperature.

The cleaning agent composition of the present invention that has the thermal deterioration suppression effect described above and/or at least one of effects (i) to (iii) described above, can be used daily for washing the human body (e.g., hair and body, including hands, face, and head), the body, including body hair, of pets such as dogs and cats, and dishes, houses, clothes, and anything in daily life. In particular, the cleaning agent composition of the present invention that has the thermal deterioration suppression effect described above and effect (ii) described above, is useful as a cleaning agent composition that can be suitably used for especially the body or hair of infants and/or people with sensitive skin.

### BRIEF DESCRIPTION OF DRAWINGS

FIG. 1-1 shows an HPLC chromatogram of a sample of the cleaning agent composition of Example 1 before storage at 50° C. (see Experimental Example 1 (2)).

FIG. 1-2 shows an HPLC chromatogram of a sample of the cleaning agent composition of Example 1 after storage at 50° C. (see Experimental Example 1 (2)).

FIG. 2-1 shows an HPLC chromatogram of a sample of the cleaning agent composition of Comparative Example 1 before storage at 50° C. (see Experimental Example 1 (2)).

FIG. 2-2 shows an HPLC chromatogram of a sample of the cleaning agent composition of Comparative Example 1 after storage at 50° C. (see Experimental Example 1 (2)).

FIG. 3 is a diagram showing a comparison of cell viability (%) in the results of a skin irritation test for various fatty acid salts (C12K: potassium laurate, C14K: potassium myristate, C16K: potassium palmitate, C18-1K: potassium oleate) in Experimental Example 3.

### DESCRIPTION OF EMBODIMENTS

### (I) Cleaning Agent Composition

The cleaning agent composition of the present invention comprises (a) fatty acid salts and (b) an SL as active ingredients. The components (a) and (b) are described below.

### (a) Fatty Acid Salts

The component (a) of the cleaning agent composition of the present invention is composed of fatty acids and bases.

The component (a) comprises a combination of (a-1) at least one member selected from the group consisting of salts of C₁₄-C₂₂ saturated fatty acids and (a-2) at least one member selected from the group consisting of salts of C₁₄-C₂₂ unsaturated fatty acids. In the present invention, the at least one member selected from the group consisting of salts of C₁₄-C₂₂ saturated fatty acids (a-1) is referred as “the component (a-1),” and the at least one member selected from the group consisting of salts of C₁₄-C₂₂ unsaturated fatty acids (a-2) is referred as “the component (a-2).”

Fatty acid salts having 12 or fewer carbon atoms are more irritating to the skin than fatty acid salts having 14 or more carbon atoms and are thus not suitable as the component (a) contained in the cleaning agent composition of the present invention. It is thus preferred that the cleaning agent composition of the present invention does not contain a fatty acid salt having 12 or fewer carbon atoms.

The component (a-1) comprises a salt of at least one saturated fatty acid selected from the group consisting of myristic acid (C14), pentadecylic acid (C15), palmitic acid (C16), isopalmitic acid (C16), margaric acid (C17), stearic acid (C18), isostearic acid (C18), arachidic acid (C20), and behenic acid (C22), all of which are C14-C22 saturated fatty acids. Salts of C14-C18 saturated fatty acids are preferred. The component (a-1) is more preferably a salt of at least one saturated fatty acid selected from the group consisting of myristic acid, palmitic acid, isopalmitic acid, stearic acid, and isostearic acid. These saturated fatty acid salts may be used singly or in any combination of two or more.

The component (a-2) comprises a salt of at least one unsaturated fatty acid selected from the group consisting of myristoleic acid (C14:1), palmitoleic acid (C16:1), oleic acid (C18:1), elaidic acid (C18:1), eicosenoic acid (C20:1), erucic acid (C22:1) (all are mono-unsaturated fatty acids), linoleic acid (C18:2), eicosadienoic acid (C20:2), docosadienoic acid (C22:2) (all are di-unsaturated fatty acids), linolenic acid (C18:3), mead acid (C20:3) (all are tri-unsaturated fatty acids), and arachidonic acid (C20:4) (tetra-unsaturated fatty acid), all of which are C14-C22 unsaturated fatty acids. Salts of C18-C20 unsaturated fatty acids are preferred. The component (a-2) is more preferably a salt of at least one C18 unsaturated fatty acid selected from the group consisting of oleic acid, linoleic acid, and linolenic acid. These unsaturated fatty acid salts may be used singly or in any combination of two or more.

As shown in Experimental Example 1, in particular, di-unsaturated fatty acid salts, tri-unsaturated fatty acid salts, and tetra-unsaturated fatty acid salts, all of which have two or more double bonds, among the components (a-2), tend to be easily decomposed by heat. Examples of unsaturated fatty acid salts that are particularly easily decomposed by heat include linoleic acid salts, linolenic acid salts, eicosadienoic acid salts, docosadienoic acid salts, mead acid salts, and arachidonic acid salts. Thus, a cleaning agent composition containing at least one of these as the component (a-2) benefits greatly from the deterioration suppression effect of the present invention.

The fatty acids constituting the component (a-1) and component (a-2) may be of natural or synthetic origin, and may be of plant or animal origin. The fatty acids constituting the component (a-1) and component (a-2) are preferably fatty acids derived from natural vegetable oils and fats.

Examples of natural oils and fats include vegetable oils and fats, such as linseed oil, perilla oil, oiticica oil, olive oil, cacao oil, kapok oil, white mustard oil, sesame oil, rice bran oil, safflower oil, shea nut oil, China wood oil, soybean oil, tea seed oil, camellia oil, corn oil, rapeseed oil, palm oil, palm kernel oil, castor oil, sunflower oil, cottonseed oil, coconut oil, Japan wax, and peanut oil; and animal oils and fats, such as horse fat, beef tallow, lard, goat fat, milk fat, fish fat, and whale oil.

Examples of counterions that react with the saturated fatty acids or unsaturated fatty acids described above to form fatty acid salts include those used as counterions in fatty acid soap. Examples of bases or basic compounds used as counterions include alkali metals, such as lithium, sodium, and potassium; alkanolamines, such as triethanolamine, diethanolamine, aminomethyl propanol, and tromethamine; basic amino acids, such as arginine and lysine; ammonium; and the like. Alkali metals, such as sodium and potassium, and alkanolamines, such as diethanolamine and triethanolamine, are preferable. Alkali metals, such as sodium and potassium, are more preferable, and potassium is particularly preferable.

The total amount of the component (a) contained in the cleaning agent composition of the present invention is 2 to 35 mass %. If the content of the component (a) is significantly less than 2 mass %, foaming tends to be poor. The lower limit of the component (a) is 2 mass % or more, preferably 3 mass % or more, more preferably 3.5 mass % or more, and even more preferably 5 mass % or more.

If the content of the component (a) is significantly greater than 35 mass %, the fluidity of the composition is poor, which makes it difficult to maintain the liquid properties. The upper limit of the component (a) is 35 mass % or less, preferably 30 mass % or less, more preferably 25 mass % or less, and even more preferably 20 mass % or less. Any combination of these lower limit values and upper limit values may be selected. The content of the component (a) in the range of, for example, 3 to 35 mass %, 3 to 30 mass %, 3.5 to 30 mass %, 3.5 to 25 mass %, or 5 to 20 mass % may be selected.

The proportion of the component (a-2) is 20 parts by mass or more based on 100 parts by mass of the component (a) contained in the cleaning agent composition of the present invention.

If the proportion of the component (a-2) is less than 20 parts by mass, the fatty acids in the resulting cleaning agent composition tend to bind to divalent metal ions in tap water, which contains divalent metal ions, to form a large amount of water-insoluble metallic soap (fatty acid calcium salts, fatty acid magnesium salts) when the cleaning agent composition is diluted or rinsed with tap water. Metallic soap tends to adhere to and remain on an object to be washed. Thus, when such a cleaning agent composition is applied to the human body (body, hair), it causes unpleasant sensations, such as a squeaky feel and skin tightening sensation after washing, and also has adverse effects, such as a decrease in skin moisture. When such a cleaning agent composition is used to wash houses, clothes, etc., it causes white residue. In contrast, by adjusting the proportion of the component (a-2) to 20 parts by mass or more based on 100 parts by mass of the component (a), the formation of metallic soap can be suppressed even when the cleaning agent composition is diluted or rinsed with tap water, and the problems of the feel during use and white residue described above can be reduced.

The tap water described above is water having a hardness of commonly 300 mg/l or less (according to the ministerial ordinance relating to the water quality standard of the Japanese Water Supply Act), typically about 10 to 100 mg/l.

The water hardness can be determined using the following formula.

$$Hardness ( mg / l ) = ( calcium amount ( mg / l ) \times 2.5 ) + ( magnesium amount ( mg / l ) \times 4 )$$

The lower limit of the proportion of the component (a-2) is 20 parts by mass or more, preferably 40 parts by mass or more, and more preferably 50 parts by mass or more, based on 100 parts by mass of the component (a). The upper limit of the proportion of the component (a-2) is not limited as long as it is less than 100 parts by mass. For example, the upper limit of the proportion of the component (a-2) is 98 parts by mass or less, preferably 95 parts by mass or less, and more preferably 93 parts by mass or less. Any combination of these lower limit values and upper limit values may be selected. The proportion of the component (a-2) in the range of, for example, 20 parts by mass to less than 100 parts by mass, 20 to 98 parts by mass, 40 to 98 parts by mass, or 50 to 95 parts by mass may be selected.

### (b) Sophorose Lipid (SL)

The component (b) used in the cleaning agent composition of the present invention is a glycolipid consisting of a hydroxyl fatty acid, and sophorose or a sophorose whose one or more hydroxyl groups are acetylated. Sophorose is a sugar consisting of two glucose molecules bound through a B1-2 bond. A hydroxyl fatty acid is a fatty acid having a hydroxyl group.

SLs are roughly classified into acidic SLs represented by the following formula (1) and lactonic SLs represented by the following formula (2). An acidic SL is a sophorose lipid in which the carboxyl group of the hydroxyl fatty acid is free. A lactonic SL is a sophorose lipid in which the carboxyl group of the hydroxyl fatty acid is bound to the sophorose in the molecule.

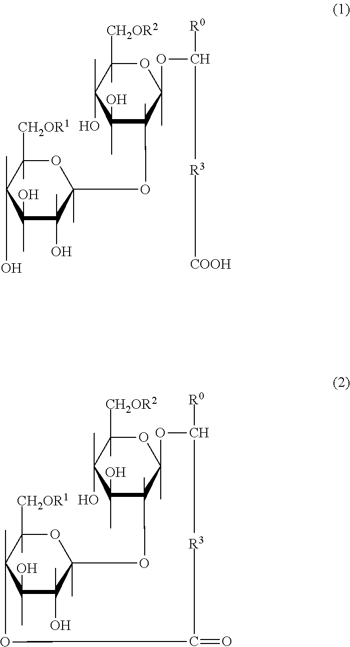

In the chemical formulas above, R⁰ is either a hydrogen atom or a methyl group. R¹ and R² are each independently a hydrogen atom or an acetyl group. R³ is composed of a saturated aliphatic hydrocarbon chain, or an unsaturated aliphatic hydrocarbon chain having at least one double bond. The aliphatic hydrocarbon chain typically has 11 to 20 carbon atoms, preferably 13 to 17 carbon atoms, and more preferably 14 to 16 carbon atoms. The SL referred to herein includes a salt of an acidic SL represented by formula (1) above. Examples of the salt include salts of alkali metals, such as potassium and sodium, salts of alkaline earth metals, such as magnesium and calcium, and ammonium salts. Salts of alkali metals are preferable, and potassium salts and sodium salts are more preferable.

The term “sophorose lipid (SL)” as used herein does not distinguish between lactonic SLs, acidic SLs, and salts thereof. In this case, the sophorose lipid (SL) may be a lactonic SL, an acidic SL, or a salt of acidic SL, or may comprise a mixture of two or more of these. When the term “sophorose lipid (SL)” refers to either of them, it is referred to as an “acidic SL” or a “lactonic SL.” Unless otherwise stated, the meaning of the term “acidic SL” includes both an acidic SL in free form and an acidic SL in salt form. Acidic SLs and lactonic SLs each may be a single compound in which each of R⁰ to R³ is a specific atom or substituent in the above formulas, or may be a mixture (composition) of multiple compounds having various R⁰ to R³.

Specifically, SLs are obtained by culturing microorganisms, especially yeasts. SLs obtained by yeast fermentation include both the acidic SLs and lactonic SLs described above.

Examples of SL-producing yeasts include Starmerella (Candida) bombicola, C. apicola, C. petrophilum, Rhodotorula (Candida) bogoriensis, and the like. By culturing such a yeast using a medium containing a high concentration of a sugar and an oily substrate, a large amount (100 to 150 g/L) of SLs can be produced and accumulated in the medium. The SLs can be separated by subjecting the culture to purification treatment, such as centrifugation, decantation, or ethyl acetate extraction, and can be obtained as a dark-brown candy-like substance by further washing the SLs with hexane. Since SLs have a greater specific gravity than water, they can be easily removed in such a manner that the culture is allowed to stand after completion of culturing to thereby separate the SLs by settling into the lower layer. The SLs thus obtained are SLs with a water content of about 50 mass %. The obtained SLs are a mixture of multiple compounds represented by formulas (1) and (2) described above. These SLs include acidic and lactonic SLs.

In the present invention, the component (b) may be an acidic SL, a lactonic SL, or a mixture of both. From the viewpoint of product stability (e.g., pH stability and suppression of a change in appearance (such as precipitation) associated with a pH change), the component (b) is preferably an acidic SL.

An acidic SL can be prepared by subjecting a mixture of an acidic SL and a lactonic SL obtained by the method described above to alkaline treatment and hydrolyzing ester linkages in the lactonic SL. Examples of alkaline treatment include an alkaline reflux method (e.g., JP2006-070231A); however, the alkaline treatment is not limited to this method, and known alkaline treatment methods can be used. For the sake of convenience, commercially available acidic SLs can be used. For example, an acidic SL is sold, for example, from Saraya Co., Ltd., under the trade name SOFORO (registered trademark) AC-30. This product is an acidic SL with a water content of 70 mass %.

The proportion of the component (b) in the cleaning agent composition of the present invention is preferably adjusted to 3 parts by mass or more per 100 parts by mass of the component (a-2) in the component (a) contained in the cleaning agent composition.

If the content of the component (b) is less than 3 parts by mass per 100 parts by mass of the component (a-2), the deterioration of the unsaturated fatty acid contained in the cleaning agent composition due to heat over time cannot be suppressed. In contrast, by incorporating the component (b) in an amount of 3 parts by mass or more per 100 parts by mass of the component (a-2) in the cleaning agent composition, the decomposition of the component (a-2) by heat can be suppressed, and as a result, causes of deterioration, such as the formation of low-molecular-weight compounds, coloring, and/or odor generation, can be eliminated or reduced.

The lower limit of the proportion of the component (b) per 100 parts by mass of the component (a-2) is 3 parts by mass or more, preferably 5 parts by mass or more, more preferably 10 parts by mass or more, and even more preferably 15 parts by mass or more. The upper limit of the proportion of the component (b) is not particularly limited from the viewpoint of the effects described above, and is, for example, 500 parts by mass or less, preferably 300 parts by mass or less, and more preferably 250 parts by mass or less, from the viewpoint of economy. Any combination of these lower limit values and upper limit values may be selected. The proportion of the component (b) in the range of, for example, 3 to less than 500 parts by mass, 5 to 300 parts by mass, or 10 to 300 parts by mass, may be selected.

As long as the above conditions are met, the proportion of the component (b) based on the cleaning agent composition of the present invention taken as 100 mass % is not limited, and can be selected from the range of 0.01 to 30 mass %.

The proportion of the component (b) in the cleaning agent composition of the present invention is preferably adjusted such that the proportion of the component (b) is 80 parts by mass or less per 100 parts by mass of the total amount of the component (a) contained in the cleaning agent composition. The proportion of the component (b) is more preferably 70 parts by mass or less, and even more preferably 60 parts by mass or less. If the proportion of the component (b) is more than 80 parts by mass per 100 parts by mass of the total amount of the component (a), the foamability, foam firmness, and storage stability of the cleaning agent composition tend to decrease.

The cleaning agent composition of the present invention is preferably adjusted to have a pH in the range of 9 to 11 by using a pH adjuster in order to exhibit good foamability and foam firmness and to reduce skin irritation. Examples of pH adjusters include acids, alkali metal salts, and the like. Examples of acids include, but are not limited to, organic acids, such as citric acid, malic acid, and succinic acid; and inorganic acids, such as sulfuric acid and hydrochloric acid. Examples of alkali metal salts include alkali metal salts, such as potassium hydroxide, sodium hydroxide, and potassium carbonate.

### (c) Water

The cleaning agent composition of the present invention is a liquid composition comprising (c) water in addition to the components (a) and (b) described above. The content of the water (c) can be selected from the range of 60 to 97 mass %. The term “liquid state” or “liquid” as used herein means that it has fluidity at a temperature of at least 5 to 50° C.

The water used in the present invention may be any water that does not impair the effects of the present invention Preferred examples include purified water, distilled water, ion-exchanged water, and RO water. When the tap water described above is used as the water, it is preferable that the tap water is used in combination with a chelating agent to sequester metal ions contained in the tap water. The amount of the chelating agent used is not limited as long as metal ions contained in the tap water can be sequestered. The amount of the chelating agent may be, for example, about 0.1 mass %; however, the amount of the chelating agent is not limited thereto.

### (d) Optional Components

The cleaning agent composition of the present invention may contain, for example, the pH adjuster described above, a thickener (viscous agent), a humectant (moisturizing agent), a chelating agent, an antiphlogistic agent, a perfume, a pigment, an antioxidant, a preservative, and/or a disinfectant, as an optional component, in addition to the components (a) to (c) described above, according to the purpose or use as long as the effects of the present invention are not impaired.

Thickeners (viscous agents) can be used to increase the viscosity of the cleaning agent composition. A wide variety of thickeners (viscous agents) that can be generally added to cleaning agents can be used as long as the effects of the present invention are not impaired. Examples include xanthan gum, guar gum, cationized guar gum, gelatin, hydroxypropyl cellulose, hydroxyethyl cellulose, cationized cellulose, and like modified celluloses, bentonite, carboxy vinyl polymers (carbomer), sodium polyacrylate, polyethylene glycol, polyvinyl alcohol, polyglutamic acid, acrylic acid-alkyl methacrylate copolymers, alkyl acrylate copolymers, and the like. These may be suitably used singly or in a combination of two or more.

Humectants (moisturizing agents) act to retain water. Humectants (moisturizing agents) can also be used to make the components of the cleaning agent composition readily compatible with each other and prevent drying of the cleaning agent composition itself. Further, humectants (moisturizing agents) can also be used to hydrate and soften an object to be washed (e.g., human skin, human hair, and clothes). Any humectant can be used as long as the effects of the present invention are not impaired. Specific examples include polyhydric alcohols, such as glycerin, butylene glycol, propanediol, hexylene glycol, dipropylene glycol, propylene glycol, hexanediol, pentanediol, octanediol, decanediol, diglycerin, and ethylhexylglycerin; amino acids, such as taurine, glutamic acid, glycine, leucine, serine, valine, threonine, alanine, isoleucine, allantoin, phenylalanine, arginine, proline, and tyrosine; and extracts of natural origin, such as perilla extract, rosemary extract, royal jelly extract, and placenta extract. Natural moisturizing factors (NMFs) are also usable. Polyhydric alcohols are preferable.

Chelating agents can be used to sequester metal ions contained in the cleaning agent composition to prevent obstructions due to metal ions (e.g., decreased foaming). Any chelating agent can be used as long as the effects of the present invention are not impaired. Specific examples include salts of organic aminocarboxylic acids, such as EDTA, NTA, DTPA, GLDA, HEDTA, GEDTA, TTHA, HIDA, and DHEG.

Examples of antiphlogistic agents include, but are not limited to, allantoin, dipotassium glycyrrhizate, and the like.

Perfumes can be used to mask the raw material odor or improve preference. Such a perfume may be composed of a single perfume component or may be a composition containing multipole perfume components. Any perfume can be used as long as the effects of the present invention are not impaired. Specific examples include synthetic perfumes, such as alcohol-based perfumes, aldehyde-based perfumes, ether-based perfumes, ester-based perfumes, ketone-based perfumes, and lactone-based perfumes; and natural perfumes derived from plants. The natural perfumes include, but are not limited to, perfumes prepared from essential oils, or distillates or fractions of plants. Such essential oils are obtained using a plant (e.g., flower, leaf, fruit, pericarp, bark, root, seed) as a raw material.

The cleaning agent composition of the present invention can be prepared by mixing the component (a), the component (b), and the component (c) together. The cleaning agent composition of the present invention can also be prepared by, if necessary, incorporating the optional component (d) described above in addition to these components. In the process of preparing the cleaning agent composition, instead of using (adding and incorporating) the component (a) itself, the component (a) may be produced by stirring fatty acids constituting the component (a) and hydroxides of salts constituting the component (a) under heating conditions to carry out neutralization reaction. The cleaning agent composition prepared in such a manner can be adjusted to have a pH in the range of 9 to 11 using a pH adjuster, as described above, and provided as the cleaning agent composition of the present invention.

### (II) Method for Suppressing Thermal Deterioration of Cleaning Agent Composition

The present invention provides a method for suppressing thermal deterioration of a cleaning agent composition comprising (a) fatty acid salts.

The fatty acid salts (a) comprise (a-1) at least one member selected from the group consisting of salts of C₁₄-C₂₂ saturated fatty acids and (a-2) at least one member selected from the group consisting of salts of C₁₄-C₂₂ unsaturated fatty acids.

This method can be performed by incorporating (b) an SL in amount of 3 parts by mass or more per 100 parts by mass of the component (a-2) in the cleaning agent composition.

If the content of the component (b) is significantly less than 3 parts by mass per 100 parts by mass of the component (a-2), the deterioration of the unsaturated fatty acid salt contained in the cleaning agent composition due to heat over time cannot be suppressed. In contrast, by incorporating the component (b) in an amount of 3 parts by mass or more per 100 parts by mass of the component (a-2) in the cleaning agent composition, the decomposition of the component (a-2) due to heat can be suppressed, and as a result, causes of deterioration, such as the formation of low-molecular-weight compounds, coloring, and/or odor generation, can be eliminated or reduced.

The component (a-2) referred to herein comprises at least an unsaturated fatty acid salt that is easily deteriorated by heat. Examples of such unsaturated fatty acid salts include unsaturated fatty acid salts having two or more double bonds in the molecule. Specific examples include linoleic acid salts, linolenic acid salts, eicosadienoic acid salts, docosadienoic acid salts, mead acid salts, and arachidonic acid salts.

The lower limit of the proportion of the component (b) is 3 parts by mass or more, preferably 5 parts by mass or more, more preferably 10 parts by mass or more, and even more preferably 15 parts by mass or more, per 100 parts by mass of the component (a-2). The upper limit of the proportion of the component (b) is not particularly limited from the viewpoint of the effects described above, and is, for example, 500 parts by mass or less, preferably 300 parts by mass or less, and more preferably 250 parts by mass or less, from the viewpoint of economy. Any combination of these lower limit values and upper limit values may be selected. The proportion of the component (b) in the range of, for example, 3 to less than 500 parts by mass, 5 to 300 parts by mass, or 10 to 300 parts by mass, may be selected.

As long as the above conditions are met, the proportion of the component (b) based on the cleaning agent composition of the present invention taken as 100 mass % is not limited, and can be selected from the range of 0.01 to 30 mass %.

In the method of the present invention, the types of component (a-1), component (a-2), and component (b), and the amounts of component (a-1) and component (a-2) are as described in section (I), and the descriptions in section (I) can be incorporated herein by reference.

### EXAMPLES

Examples are given below to illustrate the present invention in more detail. However, the present invention is not limited to these specific examples, and various modifications may be added without departing from the scope of the present invention. Unless otherwise specified, the operations in the following Experimental Examples are performed at room temperature and atmospheric pressure. “Room temperature” means a temperature in the range of 10 to 40° C.

### Reference Production Example 1: Preparation of Sophorose Lipid

A liquid medium containing, per liter, 10 g of aqueous glucose (produced by Nihon Shokuhin Kako Co., Ltd., product name: Nisshoku Gansui Kessho Budoto), 10 g of peptone (produced by Oriental Yeast Co., Ltd., product name: Peptone CB90M), and 5 g of a yeast extract (produced by Asahi Food & Healthcare Co., Ltd., product name: Meast Powder N) was used as a culture medium. Candida bombicola ATCC 22214 was cultured in the medium while shaking at 30° C. for 2 days. This was used as a pre-culture broth.

The pre-culture broth was inoculated in an amount of 4 mass % into a main culture medium (3 L) in a 5-liter fermenter, and then cultured at 30° C. at an aeration rate of 0.6 vvm for 6 days for fermentation. The main culture medium contained, per liter, 100 g of aqueous glucose, 50 g of palm olein (produced by NOF Corporation, product name: Palmary 2000), 50 g of oleic acid (produced by Acid Chem, product name: Palmac 760), 1 g of sodium chloride, 10 g of monopotassium phosphate, 10 g of magnesium sulfate heptahydrate, 2.5 g of a yeast extract (produced by Asahi Food & Healthcare Co., Ltd., product name: Meast Powder N), and 1 g of urea (pH of 4.5 to 4.8 before sterilization).

On the 6th day from the start of culturing, the fermentation was stopped. The culture broth removed from the fermenter was heated, then returned to room temperature and allowed to stand for 2 to 3 days. As a result, the culture broth was separated into the following three layers in this order from the bottom: a liquid brown precipitate layer, a milky-white solid layer presumably mainly containing cells, and a supernatant. After the supernatant was removed, industrial water or groundwater was added in an amount equal to the amount of the supernatant removed. While the resulting mixture was stirred, a 48 mass % aqueous sodium hydroxide solution was gradually added to adjust the pH to 6.5 to 6.9, thus solubilizing SLs contained in the culture broth. The resulting product was centrifuged with a tabletop centrifuge (Westfalia: produced by Westfalia Separator AG) to precipitate milky-white solids, and a supernatant was collected. While the collected supernatant was stirred, a 62.5 mass % aqueous sulfuric acid solution was gradually added to adjust the pH to 2.5 to 3.0, thus insolubilizing SLs again. After the resulting mixture was allowed to stand for 2 days, the supernatant was removed by decantation as much as possible, thus obtaining the residue as sophorose lipids (SLs) (water-containing product having an SL content of 50 mass %). The SL-containing aqueous solution contains an acidic SL and a lactonic SL in a ratio of 30:70 (mass ratio).

### Reference Production Example 2: Preparation of Acidic Sophorose Lipid

The SL-containing aqueous solution (water-containing product having an SL content of 50 mass %) obtained in Reference Production Example 1 above was adjusted to have a pH of 14 by adding an aqueous sodium hydroxide solution and treated at 80° C. for 2 hours to perform hydrolysis (alkali hydrolysis). Subsequently, the hydrolysate was returned to room temperature, and then adjusted to have a pH of 11 by using an aqueous sulfuric acid solution (9.8 M). The resulting insoluble matter was removed by filtration to obtain a filtrate as an acidic SL-containing aqueous solution. The acidic SL-containing aqueous solution contains an acidic SL in an amount of 30 mass % (water-containing product having an acidic SL content of 30 mass %).

### Experimental Example 1: Preparation and Evaluation of Cleaning Agent Compositions

### 1. Preparation of Cleaning Agent Compositions

Cleaning agent compositions (Example 1 and Comparative Example 1) were prepared by mixing the components other than pH adjusters in the proportions shown in Table 1 and finally adjusting the pH to 10 or near 10 using the pH adjusters. As the SL of the component (b), the acidic SL-containing aqueous solution prepared in Reference Production Example 2 above (water-containing product having an acidic SL content of 30 mass %) was used.

TABLE 1

| | | | | Compar- |
|---|---|---|---|---|
| | | | Exam- | ative |
| | | | ple 1 | Example 1 |
| (a) | (a-1) | Potassium palmitate | 0.5 | 0.5 |
| | | Potassium stearate | 0.5 | 0.5 |
| | (a-2) | Potassium oleate | 8.0 | 8.0 |
| | | Potassium linoleate | 1.0 | 1.0 |
| (b) | | Acidic SL-containing aqueous | 1 (0.3) | — |
| | | solution (water-containing product | | |
| | | having an acidic SL content | | |
| | | of 30 mass %) (in terms of SL) | | |
| | | Citric acid (pH adjuster)* | Appro- | Appro- |
| | | | priate | priate |
| | | | amount | amount |
| | | Potassium hydroxide (pH | Appro- | Appro- |
| | | adjuster)* | priate | priate |
| | | | amount | amount |
| (c) | | Purified water | Balance | Balance |
| Total amount | | | 100 | 100 |
| Propor- | A | Content of component (a) (%) | 10 | 10 |
| tion | B | Proportion of component (a-2) (%) | 90 | 90 |
| | | based on component (a) taken | | |
| | | as 100% | | |
| | C | Proportion of component (b) | 3.3 | 0 |
| | | (parts) per 100 parts of | | |
| | | component (a-2) | | |
| | | Content of component (b) (%) | 0.3 | 0 |
| | | Proportion of component (b) | 3.0 | 0 |
| | | (parts) per 100 parts of | | |
| | | component (a) | | |

*Amounts of pH adjusters: such amounts that the pH of the final composition becomes in the range of 9.5 to 10.5 (appropriate amounts)

### 2. Evaluation of Thermal Stability of Cleaning Agent Compositions

Each of the cleaning agent compositions prepared above (Example 1 and Comparative Example 1) was individually placed in a PET container (50 mL) and allowed to stand under dark conditions at 50° C. for 1 month. After storage, the temperature was returned to room temperature (20° C.), and the resulting samples after storage at 50° C. were subjected to appearance (hue) evaluation and HPLC analysis by the following methods. The thermal stability was evaluated based on the results.

### (1) Appearance (Hue) Evaluation

The appearance (hue) of each of the cleaning agent compositions (Example 1 and Comparative Example 1) before and after storage at 50° C. was compared. As a result, the cleaning agent composition containing no SL (Comparative Example 1) turned yellow (colored) when sored at 50° C. for 1 month. In contrast, the SL-containing cleaning agent composition (Example 1) showed no difference in hue compared to before storage. These results confirmed that incorporating an SL into a cleaning agent composition containing a saturated fatty acid salt and an unsaturated fatty acid salt suppresses coloring that occurs under heating conditions.

### (2) HPLC Analysis Evaluation

Samples of each of the cleaning agent compositions (Example 1 and Comparative Example 1) before and after storage at 50° C. were subjected to HPLC under the following conditions.

### HPLC Conditions

HPLC apparatus: Prominece-i 2030 (produced by Shimadzu Corporation)
Column: Inersil ODS-3 (φ4.6×150 mm) (produced by GL Sciences)
Mobile phase: acetonitrile:purified water:formic acid=70:30:0.1 (volume ratio)
Flow rate: 1.0 ml/min
Column temperature: 40° C.
Detection: UV 215 nm

Preliminary analysis using standard products of unsaturated fatty acids (linoleic acid and oleic acid) confirmed that the retention time of linoleic acid was 25.5 minutes under the above HPLC conditions and that the retention time of oleic acid was 42.5 minutes under the above HPLC conditions.

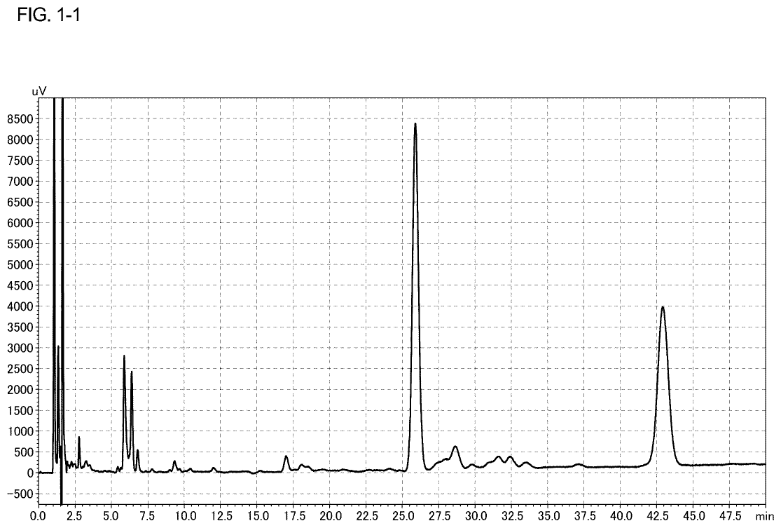

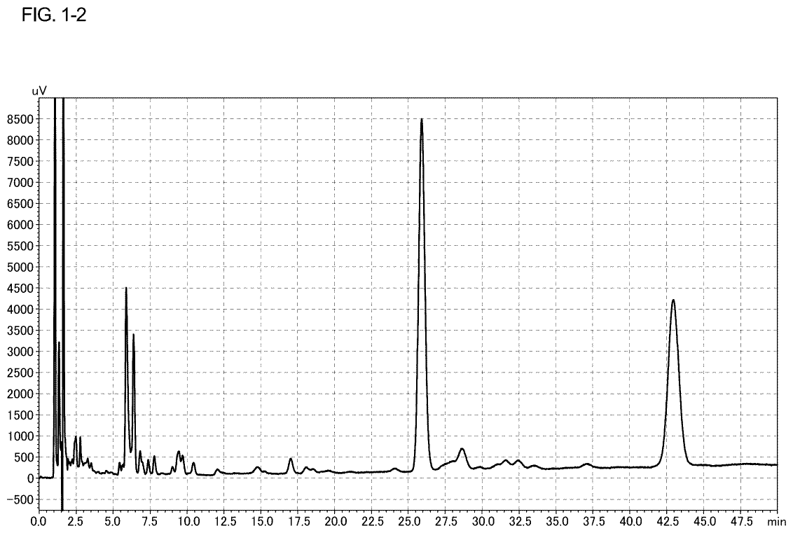

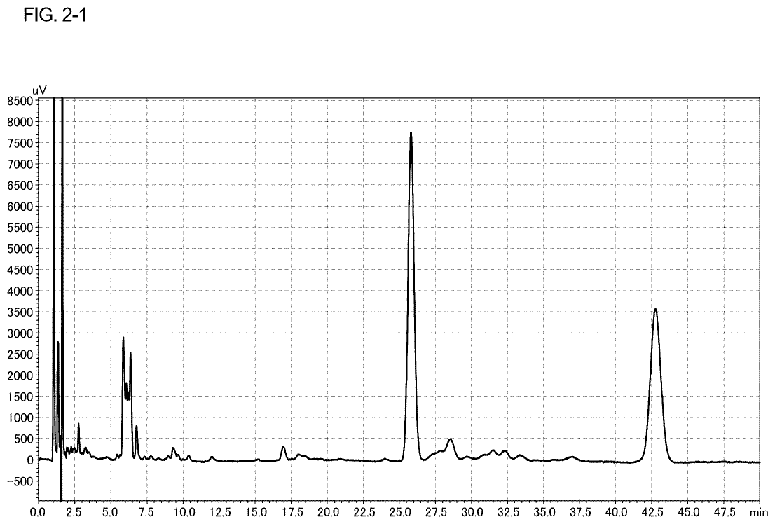

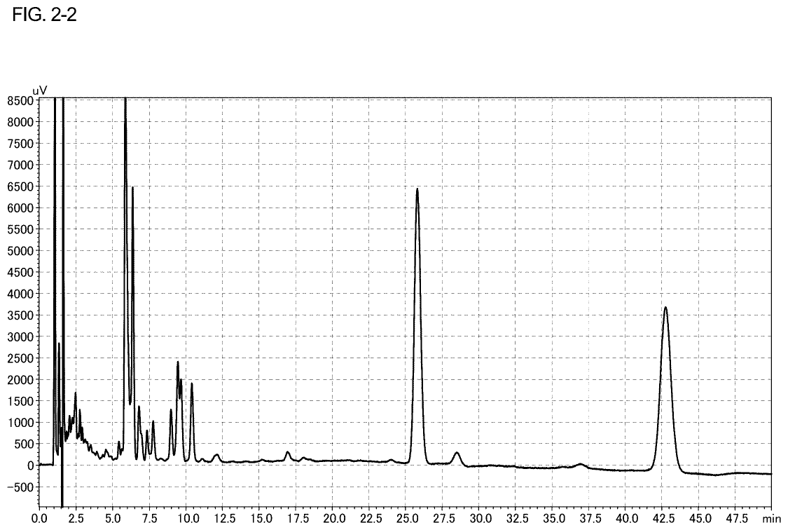

FIGS. 1-1 and 1-2 show HPLC chromatograms of the samples of the cleaning agent composition (Example 1) before and after storage at 50° C., respectively. FIGS. 2-1 and 2-2 show HPLC chromatograms of the samples of the cleaning agent composition (Comparative Example 1) before and after storage at 50° C., respectively.

Table 2 shows the change in linoleic acid content (%) before and after storage at 50° C., calculated from the peak area of linoleic acid determined from the chromatogram of each sample.

TABLE 2

| | Change in linoleic |
|---|---|
| | acid content before and |
| | after storage at 50° C. |
| Comparative Example 1: after | 82.9% *¹ |
| storage at 50° C. | |
| Example 1: after storage at | 95.5% *² |
| 50° C. | |

*¹Change relative to linoleic acid content in sample of Comparative Example 1 before storage at 50° C. taken as 100 mass %
*²Change relative to linoleic acid content in sample of Example 1 before storage at 50° C. taken as 100 mass %

As shown in Table 2, the peak of linoleic acid in the cleaning agent composition containing no SL (Comparative Example 1) was reduced by 17.1% when the composition was stored at 50° C. In addition, as shown in FIG. 2-2, in the chromatogram of the sample stored at 50° C., several peaks of low-molecular-weight compounds thought to be decomposition products of linoleic acid were observed at the retention time of 0 to 12.5 minutes. In contrast, it was confirmed that the decomposition of linoleic acid in the SL-containing cleaning agent composition (Example 1) was significantly suppressed even when the composition was stored at 50° C. (see FIG. 1-2).

### Experimental Example 2: Preparation and Evaluation of Cleaning Agent Compositions

### 1. Preparation of Cleaning Agent Compositions

Cleaning agent compositions (Examples 1 to 11 and Comparative Examples 1 to 18) were prepared by mixing the components other than pH adjusters in the proportions shown in Tables 3 to 5 and finally adjusting the pH to 10 or near 10 using the pH adjusters. As the SL of the component (b), the acidic SL-containing aqueous solution prepared in Reference Production Example 2 above (water-containing product having an acidic SL content of 30 mass %) or the SL-containing aqueous solution prepared in Reference Production Example 1 (water-containing product having an SL content of 50 mass %) was used.

### 2. Evaluation of Cleaning Agent Compositions

The cleaning agent compositions prepared above (Examples 1 to 11 and Comparative Examples 1 to 18) were evaluated for thermal stability, metallic soap formation properties, skin irritation, stability at low temperature, and foamability by the following methods.

### 2-1. Evaluation of Thermal Stability

The thermal stability of each cleaning agent composition was evaluated by storing the cleaning agent composition under heating conditions at 50° C. and under conditions at −5° C. each for a predetermined period of time, and comparing, between the two cases, (1) the content of an unsaturated fatty acid (linoleic acid), (2) the hue of the cleaning agent composition, and (3) the smell. Preliminary tests confirmed that each cleaning agent composition was stable after storage at −5° C. for 4 weeks, with no significant changes in the content of the unsaturated fatty acid (linoleic acid), hue, or smell.

### (1) Measurement of Content of Unsaturated Fatty Acid (Linoleic Acid): Evaluation of Effect of Suppressing Deterioration of Unsaturated Fatty Acid Salt

Each of the cleaning agent compositions (Examples 1 to 11 and Comparative Examples 1 to 18) was divided into two groups, i.e., a group for storage at 50° C. and a group for storage at −5° C. Samples of the group for storage at 50° C. and the group for storage at −5° C. were each placed in a PET container (50 mL) and then allowed to stand under dark conditions for 4 weeks at 50° C. and −5° C., respectively. After storage, the resulting samples of the groups (sample stored at 50° C. and sample stored at −5° C.) were subjected to HPLC under the same conditions as in Experimental Example 1 (2) to analyze the content of the unsaturated fatty acid (linoleic acid).

Based on the chromatograms obtained for the sample stored at −5° C. and the sample stored at 50° C. of each cleaning agent composition, the peak areas of linoleic acid in both samples were compared, and the residual percentage of linoleic acid in the sample stored at 50° C. was determined using the following formula. Since linoleic acid in each cleaning agent composition is stable under −5° C. storage conditions as described above, the presence or absence of deterioration of the unsaturated fatty acid in each cleaning agent composition under heating conditions (thermal stability) can be determined from the residual percentage of linoleic acid in the sample stored at 50° C. obtained using the following formula. It can be evaluated that the higher the residual percentage, the higher the effect of suppressing thermal decomposition of the unsaturated fatty acid (thermal stability effect).

$$residual percentage ( \% ) = ( A / B ) \times 100 \qquad Formula$$

A: Peak area of linoleic acid in sample stored at 50° C.
B: Peak area of linoleic acid in sample stored at −5° C.

From the residual percentage obtained using the above formula, the effect of suppressing deterioration of the unsaturated fatty acid salt was evaluated based on the following criteria.

### Criteria for Evaluating Effect of Suppressing Deterioration of Unsaturated Fatty Acid Salt

⊚: a residual percentage of 90% or more
∘: a residual percentage of 85% or more and less than 90%
x: a residual percentage of less than 85%

### (2) Measurement of Change in Hue: Evaluation of Coloring Suppression Effect

Five trained expert panelists visually compared the hues of the sample stored at −5° C. and sample stored at 50° C. of each cleaning agent composition and answered whether there was a difference between the hues of both samples. As described above, each cleaning agent composition does not change in hue when stored at −5° C. Thus, when the hue of the sample stored at 50° C. was not different from the hue of the sample stored at −5° C., it can be evaluated that the effect of suppressing the hue change (coloring) caused by heating (coloring suppression effect) is high.

From the number of panelists who answered that the hue of the sample stored at 50° C. was not different from the hue of the sample stored at −5° C., the coloring suppression effect of each cleaning agent composition was evaluated based on the following criteria.

### Criteria for Evaluating Coloring Suppression Effect

⊚: The number of panelists who answered that there was no difference was 5.
∘: The number of panelists who answered that there was no difference was 4.
Δ: The number of panelists who answered that there was no difference was 2 to 3.
x: The number of panelists who answered that there was no difference was 1 or less.

### (3) Measurement of Smell Change: Evaluation of Smell Change Suppression Effect

Five trained expert panelists compared the smells of the sample stored at −5° C. and sample stored at 50° C. of each cleaning agent composition and answered whether there was a difference between the smells of both samples. As described above, each cleaning agent composition does not change in smell when stored at −5° C. Thus, when the smell of the sample stored at 50° C. was not different from the smell of the sample stored at −5° C., it can be evaluated that the effect of suppressing the smell change caused by heating (smell change suppression effect) is high.

From the number of panelists who answered that the smell of the sample stored at 50° C. was not different from the smell of the sample stored at −5° C., the odor generation suppression effect of each cleaning agent composition was evaluated based on the following criteria.

### Criteria for Evaluating Odor Generation Suppression Effect

⊚: The number of panelists who answered that there was no difference was 5.
∘: The number of panelists who answered that there was no difference was 4.
Δ: The number of panelists who answered that there was no difference was 2 to 3.
x: The number of panelists who answered that there was no difference was 1 or less.

### 2-2. Evaluation of Metallic Soap Formation Suppression Effect

The metallic soap formation suppression effect of each cleaning agent composition was evaluated based on the turbidity of the liquid resulting from adding calcium chloride to each cleaning agent composition. Specifically, a 1 mM calcium chloride aqueous solution was added to each cleaning agent composition so that the total concentration of the fatty acid salts was 2 mM. After stirring, the turbidity of the liquid was converted to transmittance by measuring absorbance at 550 nm. It can be evaluated that the lower the transmittance, the higher the amount of metallic soap formed and the higher the metallic soap formation properties (the lower the metallic soap formation suppression effect), whereas the higher the transmittance, the lower the amount of metallic soap formed and the less likely metallic soap is to be formed (the higher the metallic soap formation suppression effect).

From the measured transmittance, the metallic soap formation suppression effect of each cleaning agent composition was evaluated based on the following criteria.

### Criteria for Evaluating Metallic Soap Formation Suppression Effect

⊚: a transmittance of 50% or more
∘: a transmittance of 40% or more to less than 50%
x: a transmittance of less than 40%

### 2-3. Evaluation of Skin Irritation

A skin irritation test was conducted based on OECD Test Guideline TG439 (in vitro skin irritation: reconstructed human epidermis test method).

After each cleaning agent composition was prepared, 25 μL of the cleaning agent composition was added to a three-dimensional cultured skin model (LabCyte EPI-MODEL 24 produced by J-TEC). After 15 minutes of exposure, the model was washed with PBS to remove the cleaning agent composition. After 42 hours of culture, culture was performed again in a medium supplemented with MTT, and living cells were stained. Subsequently, the dye was extracted with isopropanol, and the absorbance (570 nm) of the extract was measured. As a negative control, a test was conducted in the same manner, using purified water in place of the cleaning agent composition as a test sample.

The cell viability of cultured skin to which each cleaning agent composition was added was calculated using the following formula.

$$Cell viability ( \% ) = ( A / B ) \times 100$$

A: Average absorbance measured using each cleaning agent composition as a test sample
B: Average absorbance measured using purified water as a test sample (negative control)

Based on OECD TG439, the skin irritation of each cleaning agent composition was evaluated according to the following criteria.

### Criteria for Evaluating Skin Irritation

⊚ Non-irritating: The cell viability was 50% or more.
x Irritating: The cell viability was less than 50%.

### 2-4. Stability at Low Temperature

After each cleaning agent composition was prepared, the cleaning agent composition was placed in a PET container (50 mL) and stored under dark conditions at 5° C. for 4 weeks, and the condition of the liquid of the cleaning agent composition was visually observed over time to evaluate the presence or absence of precipitation, floating matter, and solid-liquid separation. The stability at low temperature was evaluated based on the following criteria.

### Criteria for Evaluating Stability at Low Temperature

⊚: During the 4-week storage period, no precipitation, floating matter, or solid-liquid separation was observed, and the components were homogeneously dissolved.
∘: During the 1-week storage period, no precipitation, floating matter, or solid-liquid separation was observed, and the components were homogeneously dissolved. However, after 1 week of storage, precipitation, floating matter, or solid-liquid separation was observed.
x: During the 1-week storage period, precipitation, floating matter, or solid-liquid separation was observed.

### 2-5. Foamability (Foaming Properties)

2.5 mL of a sample and 2.5 mL of tap water were placed in a test tube (18 mm in diameter×180 mm in length) and stirred with a vortex mixer for 20 seconds. The amount of bubbles (the length from the liquid surface to the top of bubbles in the test tube (foam height)) of the sample was measured immediately after stirring. This test was performed at room temperature (25±5° C.).

### Criteria for Evaluating Foamability

∘: The foam height immediately after stirring was 10 mm or more.
x: The foam height immediately after stirring was less than 10 mm.

Tables 3 to 5 show the formulation and evaluation results of each of the cleaning agent compositions (Examples 1 to 11 and Comparative Examples 1 to 18).

TABLE 3

| | | | Ex. 1 | Ex. 2 | Ex. 3 | Ex. 4 | Ex. 5 | Ex. 6 |
|---|---|---|---|---|---|---|---|---|
| (a) | (a-1) | Potassium caprate | — | — | — | — | — | — |
| | | Potassium laurate | — | — | — | — | — | — |
| | | Potassium myristate | — | 4.0 | 4.0 | 2.3 | 2.3 | 4.0 |
| | | Potassium palmitate | 0.5 | 0.4 | 0.4 | 0.4 | 0.4 | 0.4 |
| | | Potassium stearate | 0.5 | — | — | 0.1 | 0.1 | — |
| | (a-2) | Potassium oleate | 8.0 | 4.8 | 4.8 | 0.8 | 0.8 | 4.8 |
| | | Potassium linoleate | 1.0 | 0.8 | 0.8 | 0.1 | 0.1 | 0.8 |
| (b) | | Acidic SL-containing aqueous | 1 (0.3) | 1 (0.3) | 3 (0.9) | 5 (1.5) | 6 (1.8) | — |
| | | solution (water-containing | | | | | | |
| | | product having an acidic SL | | | | | | |
| | | content of 30 mass %) (in terms | | | | | | |
| | | of SL) | | | | | | |
| | | SL-containing aqueous solution | — | — | — | — | — | 2 (1) |
| | | (water-containing product | | | | | | |
| | | having an SL content of 50 | | | | | | |
| | | mass % t) (in terms of SL) | | | | | | |
| (d) | | Glycerin | — | 5.0 | 10.0 | 10.0 | 10.0 | — |
| | | Hexylene glycol | — | — | — | — | — | — |
| | | Perfume (including essential oil) | — | 0.05 | 0.2 | — | — | 0.5 |
| | | Citric acid (pH adjuster)* | Appropriate | Appropriate | Appropriate | Appropriate | Appropriate | Appropriate |
| | | | amount | amount | amount | amount | amount | amount |
| | | Potassium hydroxide (pH | Appropriate | Appropriate | Appropriate | Appropriate | Appropriate | Appropriate |
| | | adjuster)* | amount | amount | amount | amount | amount | amount |
| | | Xanthan gum | — | — | — | — | — | 0.3 |
| | | Edetic acid salt | — | — | — | — | — | — |
| | | Alkyl glucoside | — | — | — | — | — | — |
| | | Polyglyceryl-2 oleate | — | — | — | — | — | — |
| | | Sodium alkyl ether sulfate | — | — | — | — | — | — |
| (c) | | Purified water | Balance | Balance | Balance | Balance | Balance | Balance |
| Total amount | | | 100 | 100 | 100 | 100 | 100 | 100 |
| Proportion | A | Content of component (a) (%) | 10 | 10 | 10 | 3.7 | 3.7 | 10 |
| | B | Proportion of component (a-2) | 90 | 56 | 56 | 24 | 24 | 56 |
| | | (%) based on component (a) | | | | | | |
| | | taken as 100% | | | | | | |
| | C | Proportion of component (b) | 3.3 | 5.3 | 16 | 167 | 200 | 17.8 |
| | | (parts) per 100 parts of | | | | | | |
| | | component (a-2) | | | | | | |
| | | Content of component (b) (%) | 0.3 | 0.3 | 0.9 | 1.5 | 1.8 | 1 |
| | | Proportion of component (b) | 3.0 | 3.0 | 9 | 40.5 | 48.6 | 10 |
| | | (parts) per 100 parts of | | | | | | |
| | | component (a) | | | | | | |
| Evaluation | Thermal | Unsaturated fatty acid salt | ⊚ | ⊚ | ⊚ | ⊚ | ⊚ | ⊚ |
| | stability | deterioration suppression effect | | | | | | |
| | | Coloring suppression effect | ◯ | ◯ | ⊚ | ⊚ | ⊚ | ⊚ |
| | | Odor generation suppression | ◯ | ◯ | ⊚ | ⊚ | ⊚ | ⊚ |
| | | effect | | | | | | |

| | | | | | | |
|---|---|---|---|---|---|---|
| Metallic soap formation suppression effect | ⊚ | ⊚ | ⊚ | ◯ | ◯ | ⊚ |
| Skin irritation | ⊚ | ⊚ | ⊚ | ⊚ | ⊚ | ⊚ |
| Stability at low temperature | ⊚ | ⊚ | ⊚ | ◯ | ◯ | ◯ |
| Foamability | ◯ | ◯ | ◯ | ◯ | ◯ | ◯ |

| | | | Ex. 7 | Ex. 8 | Ex. 9 | Ex. 10 | Ex. 11 |
|---|---|---|---|---|---|---|---|
| (a) | (a-1) | Potassium caprate | — | — | — | — | — |
| | | Potassium laurate | — | — | — | — | — |
| | | Potassium myristate | 1.5 | 12 | 15 | — | 8.0 |
| | | Potassium palmitate | 0.1 | 1.8 | 0.9 | 2.0 | 0.8 |
| | | Potassium stearate | — | — | — | — | — |
| | (a-2) | Potassium oleate | 0.35 | 15.7 | 7.5 | 15 | 9.6 |
| | | Potassium linoleate | 0.05 | 2.5 | 1.6 | 2.5 | 1.6 |
| (b) | | Acidic SL-containing aqueous | 0.6 (0.18) | 6.6 (1.98) | 2 (0.6) | 6.6 (1.98) | 3.3 (0.99) |
| | | solution (water-containing | | | | | |
| | | product having an acidic SL | | | | | |
| | | content of 30 mass %) (in terms | | | | | |
| | | of SL) | | | | | |
| | | SL-containing aqueous solution | — | — | — | — | — |
| | | (water-containing product | | | | | |
| | | having an SL content of 50 | | | | | |
| | | mass % t) (in terms of SL) | | | | | |
| (d) | | Glycerin | 5.0 | — | — | 10.0 | 5.0 |
| | | Hexylene glycol | — | 3.0 | 5.0 | 5.0 | 5.0 |
| | | Perfume (including essential oil) | — | — | — | — | — |
| | | Citric acid (pH adjuster)* | Appropriate | Appropriate | Appropriate | Appropriate | Appropriate |
| | | | amount | amount | amount | amount | amount |
| | | Potassium hydroxide (pH | Appropriate | Appropriate | Appropriate | Appropriate | Appropriate |
| | | adjuster)* | amount | amount | amount | amount | amount |
| | | Xanthan gum | — | — | — | — | 0.1 |
| | | Edetic acid salt | — | 0.1 | — | — | — |
| | | Alkyl glucoside | — | — | — | — | — |
| | | Polyglyceryl-2 oleate | — | — | — | — | — |
| | | Sodium alkyl ether sulfate | — | — | — | — | — |
| (c) | | Purified water | Balance | Balance | Balance | Balance | Balance |
| Total amount | | | 100 | 100 | 100 | 100 | 100 |
| Proportion | A | Content of component (a) (%) | 2.0 | 32 | 25 | 19.5 | 20 |
| | B | Proportion of component (a-2) | 20 | 62 | 36 | 89 | 56 |
| | | (%) based on component (a) | | | | | |
| | | taken as 100% | | | | | |
| | C | Proportion of component (b) | 45 | 10.9 | 6.6 | 11.3 | 9.0 |
| | | (parts) per 100 parts of | | | | | |
| | | component (a-2) | | | | | |
| | | Content of component (b) (%) | 0.18 | 1.98 | 0.6 | 1.98 | 0.99 |
| | | Proportion of component (b) | 9 | 6.2 | 2.4 | 10.2 | 4.95 |
| | | (parts) per 100 parts of | | | | | |
| | | component (a) | | | | | |
| Evaluation | Thermal | Unsaturated fatty acid salt | ⊚ | ⊚ | ⊚ | ⊚ | ⊚ |
| | stability | deterioration suppression effect | | | | | |
| | | Coloring suppression effect | ⊚ | ⊚ | ◯ | ◯ | ◯ |
| | | Odor generation suppression | ⊚ | ⊚ | ◯ | ◯ | ◯ |
| | | effect | | | | | |

| | | | | | |
|---|---|---|---|---|---|
| Metallic soap formation suppression effect | ◯ | ⊚ | ◯ | ⊚ | ⊚ |
| Skin irritation | ⊚ | ⊚ | ⊚ | ⊚ | ⊚ |
| Stability at low temperature | ◯ | ⊚ | ◯ | ◯ | ◯ |
| Foamability | ◯ | ◯ | ◯ | ◯ | ◯ |

*Amounts of pH adjusters: such amounts that the pH of the final composition becomes in the range of 9.5 to 10.5 (appropriate amounts)

TABLE 4

| | | | Comp. | Comp. | Comp. | Comp. | Comp. |
|---|---|---|---|---|---|---|---|
| | | | Ex. 1 | Ex. 2 | Ex. 3 | Ex. 4 | Ex. 5 |
| (a) | (a-1) | Potassium caprate | — | — | — | | |
| | | Potassium laurate | — | — | — | 4.0 | 4.8 |
| | | Potassium myristate | — | — | 3.0 | 3.0 | 3.5 |
| | | Potassium palmitate | 0.5 | 0.5 | 2.0 | — | — |
| | | Potassium stearate | 0.5 | 0.5 | 0.5 | — | — |
| | (a-2) | Potassium oleate | 8.0 | 8.0 | — | 2.0 | 3.6 |
| | | Potassium linoleate | 1.0 | 1.0 | — | 1.0 | 0.4 |
| (b) | | Acidic SL-containing aqueous | — | — | — | — | 0.3 (0.09) |
| | | solution (water-containing | | | | | |
| | | product having an acidic SL | | | | | |
| | | content of 30 mass %) (in terms | | | | | |
| | | of SL) | | | | | |
| | | SL-containing aqueous solution | — | — | — | — | — |
| | | (water-containing product having | | | | | |
| | | an SL content of 50 mass %) (in | | | | | |
| | | terms of SL) | | | | | |
| (d) | | Glycerin | — | 5.0 | — | — | 5.0 |
| | | Hexylene glycol | — | — | — | — | — |
| | | Perfume (including essential oil) | — | — | — | — | 0.2 |
| | | Citric acid (pH adjuster)* | Appropriate | Appropriate | Appropriate | Appropriate | Appropriate |
| | | | amount | amount | amount | amount | amount |
| | | Potassium hydroxide (pH | Appropriate | Appropriate | Appropriate | Appropriate | Appropriate |
| | | adjuster)* | amount | amount | amount | amount | amount |
| | | Xanthan gum | — | — | — | — | — |
| | | Edetic acid salt | — | — | — | — | — |
| | | Alkyl glucoside | — | — | — | — | — |
| | | Polyglyceryl-2 oleate | — | — | — | — | — |
| | | Sodium alkyl ether sulfate | — | — | — | — | — |
| (c) | | Purified water | Balance | Balance | Balance | Balance | Balance |
| Total amount | | | 100 | 100 | 100 | 100 | 100 |
| Proportion | A | Content of component (a) (%) | 10 | 10 | 5.5 | 10 | 12.3 |
| | B | Proportion of component (a-2) | 90 | 90 | 0 | 30 | 32.5 |
| | | (%) based on component (a) | | | | | |
| | | taken as 100% | | | | | |
| | C | Proportion of component (b) | 0 | 0 | — | 0 | 2.3 |
| | | (parts) per 100 parts of | | | | | |
| | | component (a-2) | | | | | |
| | | Content of component (b) (%) | 0 | 0 | 0 | 0 | 0.09 |
| | | Proportion of component (b) | 0 | 0 | 0 | 0 | 0.73 |
| | | (parts) per 100 parts of | | | | | |
| | | component (a) | | | | | |
| Evaluation | Thermal | Unsaturated fatty acid salt | X | X | ND | X | X |
| | stability | deterioration suppression effect | | | | | |
| | | Coloring suppression effect | X | X | ⊚ | X | X |
| | | Odor generation suppression | X | X | ⊚ | X | X |
| | | effect | | | | | |

| | | | | | |
|---|---|---|---|---|---|
| Metallic soap formation suppression effect | ⊚ | ⊚ | X | ◯ | ◯ |
| Skin irritation | ⊚ | ⊚ | ⊚ | X | X |
| Stability at low temperature | ⊚ | ⊚ | X | ◯ | ◯ |
| Foamability | ◯ | ◯ | ◯ | ◯ | ◯ |

| | | | Comp. | Comp. | Comp. |
|---|---|---|---|---|---|
| | | | Ex. 6 | Ex. 7 | Ex. 8 |
| (a) | (a-1) | Potassium caprate | | | |
| | | Potassium laurate | 7.5 | 10.4 | 18.4 |
| | | Potassium myristate | 3.0 | 2.9 | 6.5 |
| | | Potassium palmitate | 1.3 | 1.7 | 2.9 |
| | | Potassium stearate | 1.0 | — | 0.9 |
| | (a-2) | Potassium oleate | 24.0 | 2.5 | 5.6 |
| | | Potassium linoleate | 3.2 | — | 0.7 |
| (b) | | Acidic SL-containing aqueous | — | 1.2 (0.36) | — |
| | | solution (water-containing | | | |
| | | product having an acidic SL | | | |
| | | content of 30 mass %) (in terms | | | |
| | | of SL) | | | |
| | | SL-containing aqueous solution | — | — | 0.8 (0.4) |
| | | (water-containing product having | | | |
| | | an SL content of 50 mass %) (in | | | |
| | | terms of SL) | | | |
| (d) | | Glycerin | — | — | — |
| | | Hexylene glycol | — | — | — |
| | | Perfume (including essential oil) | — | — | — |
| | | Citric acid (pH adjuster)* | Appropriate | Appropriate | Appropriate |
| | | | amount | amount | amount |
| | | Potassium hydroxide (pH | Appropriate | Appropriate | Appropriate |
| | | adjuster)* | amount | amount | amount |
| | | Xanthan gum | — | — | — |
| | | Edetic acid salt | — | — | — |
| | | Alkyl glucoside | — | — | — |
| | | Polyglyceryl-2 oleate | — | — | — |
| | | Sodium alkyl ether sulfate | — | — | — |
| (c) | | Purified water | Balance | Balance | Balance |
| Total amount | | | 100 | 100 | 100 |
| Proportion | A | Content of component (a) (%) | 40 | 17.5 | 35 |
| | B | Proportion of component (a-2) | 68 | 14 | 18 |
| | | (%) based on component (a) | | | |
| | | taken as 100% | | | |
| | C | Proportion of component (b) | 0 | 14 | 6.3 |
| | | (parts) per 100 parts of | | | |
| | | component (a-2) | | | |
| | | Content of component (b) (%) | 0 | 0.36 | 0.4 |
| | | Proportion of component (b) | 0 | 2.06 | 1.14 |
| | | (parts) per 100 parts of | | | |
| | | component (a) | | | |
| Evaluation | Thermal | Unsaturated fatty acid salt | X | ND | ⊚ |
| | stability | deterioration suppression effect | | | |
| | | Coloring suppression effect | X | ⊚ | ⊚ |
| | | Odor generation suppression | X | ⊚ | ⊚ |
| | | effect | | | |

| | | | |
|---|---|---|---|
| Metallic soap formation suppression effect | ◯ | X | X |
| Skin irritation | X | X | X |
| Stability at low temperature | X | ⊚ | ◯ |
| Foamability | ◯ | ◯ | ◯ |

*Amounts of pH adjusters: such amounts that the pH of the final composition becomes in the range of 9.5 to 10.5 (appropriate amounts)
ND: Not measured (no data)

TABLE 5

| | | | Comp. | Comp. | Comp. | Comp. | Comp. |
|---|---|---|---|---|---|---|---|
| | | | Ex. 9 | Ex. 10 | Ex. 11 | Ex. 12 | Ex 13 |
| (a) | (a-1) | Potassium caprate | — | 1.2 | — | — | — |
| | | Potassium laurate | 3.8 | 8.9 | 6.0 | 3.6 | 4.2 |
| | | Potassium myristate | 0.5 | 1.7 | 1.5 | 1.2 | 3.5 |
| | | Potassium palmitate | 0.1 | — | 0.9 | 0.7 | 1.1 |
| | | Potassium stearate | — | — | — | — | — |
| | (a-2) | Potassium oleate | — | — | — | — | — |
| | | Potassium linoleate | — | — | — | — | — |
| (b) | | Acidic SL-containing aqueous solution | 2.1 (0.63) | 14 (4.2) | 3.5 (1.05) | 5.0 (1.5) | 6.7 (2.01) |
| | | (water-containing product having an acidic | | | | | |
| | | SL content of 30 mass %) (in terms of SL) | | | | | |
| | | SL-containing aqueous solution (water- | — | — | — | — | — |
| | | containing product having an SL content of | | | | | |
| | | 50 mass %) (in terms of SL) | | | | | |
| (d) | | Glycerin | 10.0 | 5 | 8 | 3 | 5 |
| | | Butylene glycol | — | 1 | — | — | — |
| | | Perfume (including essential oil) | 0.2 | 0.1 | — | — | — |
| | | Citric acid (pH adjuster)* | Appropriate | Appropriate | Appropriate | Appropriate | Appropriate |
| | | | amount | amount | amount | amount | amount |
| | | Potassium hydroxide (pH adjuster)* | Appropriate | Appropriate | Appropriate | Appropriate | Appropriate |
| | | | amount | amount | amount | amount | amount |
| (c) | | Purified water | Balance | Balance | Balance | Balance | Balance |
| Total amount | | | 100 | 100 | 100 | 100 | 100 |
| Proportion | A | Content of component (a) (%) | 4.4 | 11.8 | 8.4 | 5.5 | 8.8 |
| | B | Proportion of component (a-2) (%) based on | 0 | 0 | 0 | 0 | 0 |
| | | component (a) taken as 100% | | | | | |
| | C | Proportion of component (b) (parts) per 100 | — | — | — | — | — |
| | | parts of component (a-2) | | | | | |
| | | Content of component (b) (%) | 0.63 | 4.2 | 1.05 | 1.5 | 2.01 |
| | | Proportion of component (b) (parts) per 100 | 14 | 35.6 | 12.5 | 27.3 | 22.8 |
| | | parts of component (a) | | | | | |
| Evaluation | Thermal | Unsaturated fatty acid salt deterioration | ND | ND | ND | ND | ND |
| | stability | suppression effect | | | | | |
| | | Coloring suppression effect | ⊚ | ⊚ | ⊚ | ⊚ | ⊚ |
| | | Odor generation suppression effect | ⊚ | ⊚ | ⊚ | ⊚ | ⊚ |
| | Metallic soap formation suppression effect | | X | X | X | X | X |
| | Skin irritation | | X | X | X | X | X |
| | Stability at low temperature | | ◯ | ◯ | ◯ | ◯ | ◯ |
| | Foamability | | ◯ | ◯ | ◯ | ◯ | ◯ |
| | | | Comp. | Comp. | Comp. | Comp. | Comp. |
| | | | Ex. 14 | Ex 15 | Ex. 16 | Ex. 17 | Ex. 18 |
| (a) | (a-1) | Potassium caprate | 0.5 | — | — | — | — |
| | | Potassium laurate | 0.4 | 8.7 | 9.5 | 2.4 | 4.8 |
| | | Potassium myristate | 2.8 | 3.1 | 2.9 | 3.5 | 1.7 |
| | | Potassium palmitate | 1.0 | 1.5 | 1.7 | 0.9 | — |
| | | Potassium stearate | 0.5 | — | — | — | — |
| | (a-2) | Potassium oleate | 0.3 | 0.8 | 3.4 | 5.7 | 8.0 |
| | | Potassium linoleate | 0.1 | — | — | — | — |
| (b) | | Acidic SL-containing aqueous solution | 6.3 (1.89) | 9.0 (2.7) | 16 (4.8) | 7.0 (2.1) | 6 (1.8) |
| | | (water-containing product having an acidic | | | | | |
| | | SL content of 30 mass %) (in terms of SL) | | | | | |
| | | SL-containing aqueous solution (water- | — | — | — | — | — |
| | | containing product having an SL content of | | | | | |
| | | 50 mass %) (in terms of SL) | | | | | |
| (d) | | Glycerin | — | 15 | 4 | 4.0 | 8 |
| | | Butylene glycol | — | — | 4 | — | 1 |
| | | Perfume (including essential oil) | — | — | — | — | — |
| | | Citric acid (pH adjuster)* | Appropriate | Appropriate | Appropriate | Appropriate | Appropriate |
| | | | amount | amount | amount | amount | amount |
| | | Potassium hydroxide (pH adjuster)* | Appropriate | Appropriate | Appropriate | Appropriate | Appropriate |
| | | | amount | amount | amount | amount | amount |
| (c) | | Purified water | Balance | Balance | Balance | Balance | Balance |
| Total amount | | | 100 | 100 | 100 | 100 | 100 |
| Proportion | A | Content of component (a) (%) | 5.6 | 14.1 | 17.5 | 12.5 | 14.5 |
| | B | Proportion of component (a-2) (%) based on | 7 | 13 | 19 | 46 | 55 |
| | | component (a) taken as 100% | | | | | |
| | C | Proportion of component (b) (parts) per 100 | 472 | 338 | 141 | 37 | 23 |
| | | parts of component (a-2) | | | | | |
| | | Content of component (b) (%) | 1.89 | 2.7 | 4.8 | 2.1 | 1.8 |
| | | Proportion of component (b) (parts) per 100 | 33.7 | 19.1 | 27.4 | 16.8 | 12.4 |
| | | parts of component (a) | | | | | |
| Evaluation | Thermal | Unsaturated fatty acid salt deterioration | ⊚ | ND | ND | ND | ND |
| | stability | suppression effect | | | | | |
| | | Coloring suppression effect | ⊚ | ⊚ | ⊚ | ⊚ | ⊚ |
| | | Odor generation suppression effect | ⊚ | ⊚ | ⊚ | ⊚ | ⊚ |

| | | | | | |
|---|---|---|---|---|---|
| Metallic soap formation suppression effect | X | X | X | ◯ | ◯ |
| Skin irritation | X | X | X | X | X |
| Stability at low temperature | ◯ | ◯ | ◯ | ◯ | ◯ |
| Foamability | ◯ | ◯ | ◯ | ◯ | ◯ |

*Amounts of pH adjusters: such amounts that the pH of the final composition becomes in the range of 9.5 to 10.5 (appropriate amounts)
ND: Not measured (no data)

The cleaning agent compositions of Examples 1 to 11 contain (a-1) one or more saturated fatty acid salts having 14 or more carbon atoms and (a-2) unsaturated fatty acid salts having 14 or more carbon atoms as (a) fatty acid salts, and (b) an SL. In each cleaning agent composition, the proportion of the component (a) is within the range of 2 to 35 mass % based on the composition taken as 100 mass %, the proportion of the component (a-2) is 20 parts by mass or more based on 100 parts by mass of the total amount of the component (a), and the proportion of the component (b) is 3 parts by mass or more per 100 parts by mass of the component (a-2). As shown in Table 3, all of these cleaning agent compositions were confirmed to have low skin irritation, high thermal stability (unsaturated fatty acid salt deterioration suppression effect, coloring suppression effect, and odor generation suppression effect), and good stability at low temperature and foamability, and further to suppress metallic soap formation.

In contrast, cleaning agent compositions containing no unsaturated fatty acid salt (Comparative Examples 3 and 9 to 13) all had high metallic soap formation properties and had no metallic soap formation suppression effect. Cleaning agent compositions containing one or more unsaturated fatty acid salts in an amount of less than 20 parts by mass based on 100 parts by mass of the total amount of fatty acid salts (Comparative Examples 7, 8, and 14 to 16) also had high metallic soap formation properties and had no metallic soap formation suppression effect (Tables 4 and 5). A large amount of metallic soap formed causes a squeaky feel and skin tightening sensation when such cleaning agent compositions are used for body washing and causes white residue when such cleaning agent compositions are used for washing dishes, clothes, and houses.

In contrast, cleaning agent compositions containing one or more unsaturated fatty acid salts in an amount of 20 parts by mass or more based on 100 parts by mass of the total amount of fatty acid salts (Examples 1 to 11 and Comparative Examples 4 to 6, 17, and 18) had a high metallic soap formation suppression effect and produced less metallic soap when diluted with tap water (see Tables 3 to 5). These results confirmed that by setting the proportion of one or more unsaturated fatty acid salts to 20 parts by mass or more based on 100 parts by mass of the total amount of fatty acid salts, a cleaning agent composition having a high metallic soap formation suppression effect and producing less metallic soap can be prepared.

The cleaning agent compositions of Comparative Examples 1, 2, and 4 to 6 all had poor thermal stability, and unsaturated fatty acid deterioration, coloring, and odor generation were observed when they are stored at 50° C. In contrast, a cleaning agent composition containing no unsaturated fatty acid salt (Comparative Example 3) had good thermal stability (suppression of coloring and suppression of odor generation) (see Table 4). Considering the results of Experimental Example 1 described above, it is believed that the coloring and odor generation caused by storage at 50° C. were due to deterioration of the unsaturated fatty acid salts contained in the cleaning agent compositions (decomposition to low-molecular-weight compounds).

Cleaning agent compositions containing one or more unsaturated fatty acid salts and an SL in an amount of 3 parts by mass or more per 100 parts by mass of the one or more unsaturated fatty acid salts (Examples 1 to 11 and Comparative Examples 14 to 18) had good thermal stability (suppression of unsaturated fatty acid salt deterioration, suppression of coloring, and suppression of odor generation) (see Tables 3 and 5). In contrast, the cleaning agent composition of Comparative Example 5, which contains unsaturated fatty acid salts and an SL in an amount of 2.3 parts by mass per 100 parts by mass of the unsaturated fatty acid salts, had poor thermal stability (see Table 4).

These results confirmed that the thermal stability problems (unsaturated fatty acid deterioration, coloring, odor generation) caused by incorporating an unsaturated fatty acid salt as a fatty acid salt can be reduced or solved by incorporating an SL in an amount of 3 parts by mass or more per 100 parts by mass of the unsaturated fatty acid salt.

Cleaning agent compositions containing no fatty acid salt having less than 14 carbon atoms (Examples 1 to 11 and Comparative Examples 1 to 3) were all less irritating to the skin. In contrast, cleaning agent compositions containing one or more fatty acid salts having less than 14 carbon atoms (Comparative Examples 4 to 18) were all found to be irritating to the skin. From the results of Experimental Example 3 described below, it is believed that the skin irritation of the cleaning agent compositions was due to the fatty acid salts having less than 14 carbon atoms, in particular, the saturated fatty acid salt having 12 carbon atoms. This confirmed that a cleaning agent composition that is less irritating to the skin can be prepared by using a fatty acid salt having 14 or more carbon atoms without using a fatty acid salt having less than 14 carbon atoms as a fatty acid salt. That is, a cleaning agent composition containing no fatty acid salt having less than 14 carbon atoms is less irritating to the skin and can therefore be suitably used as a cleaning agent composition for the body or hair of infants, who have delicate skin, and people with sensitive skin.

A cleaning agent composition containing no C₁₄-C₂₂ unsaturated fatty acid salt (Comparative Example 3) and a cleaning agent composition having a fatty acid salt content of 40 mass % (Comparative Example 6) had poor stability at low temperature (Table 4). In contrast, cleaning agent compositions containing one or more C₁₄-C₂₂ unsaturated fatty acid salts (Examples 1 to 11 and Comparative Examples 1, 2, 4, 5, 7, 8, and 14 to 18), and cleaning agent compositions containing no unsaturated fatty acid salt, but containing one or more saturated fatty acid salts having less than 14 carbon atoms (Comparative Examples 9 to 13) all had good stability at low temperature (Tables 3 and 5). These cleaning agent compositions all had a fatty acid salt content of 35 mass % or less.

These results confirmed that a cleaning agent composition with good stability at low temperature can be prepared by incorporating an C₁₄-C₂₂ unsaturated fatty acid salt and adjusting the total fatty acid salt content to 35 mass % or less.

All of the cleaning agent compositions (Examples 1 to 11 and Comparative Examples 1 to 18) contained fatty acid salts in an amount of 2 mass % or more of the total amount and were confirmed to have foamability required as cleaning agent compositions.

### Experimental Example 3: Evaluation of Skin Irritation

The skin irritation of alkali metal salts of various fatty acids (potassium laurate, potassium myristate, potassium palmitate, and potassium oleate) was evaluated by the following method based on OECD Test Guideline TG439.

### (1) Evaluation Method

1) Each culture insert of a human three-dimensional cultured epidermis model is individually transferred to a well containing 0.5 mL of assay medium, and pre-cultured in a CO₂ incubator for about 24 hours.
2) 25 μL of each test sample (each 0.1 M fatty acid aqueous solution) is individually added to the surface of the cultured epidermis, followed by exposure for 15 minutes (N=3).
3) The sample in each culture insert is washed off with sterile PBS (washing is repeated 15 times or more to completely remove the test sample).
4) Each washed culture insert is transferred to a well containing 1 mL of fresh assay medium and subjected to post treatment incubation in a CO₂ incubator for 42 hours.
5) After culture, each culture insert is transferred to a well containing 0.5 mL of MTT medium and cultured in a CO₂ incubator for 3 hours.
6) The cultured epidermis in each culture insert is removed with tweezers and placed in a microtube, 300 μL of isopropanol is added thereto to completely immerse the cultured epidermis, the cultured epidermis is allowed to stand for one night or more, and the dye is extracted.
7) 200 μL of the solution in the microtube is put in each well of a 96-well plate, and the absorbance at 570 nm is measured with a plate reader.

### (2) Evaluation Results

As a negative control, the same test was performed using purified water in place of the fatty acid solutions as a test sample. The viability (%) of cells exposed to the fatty acid solutions was determined in the same manner as described in section 2-3 of Experimental Example 2.

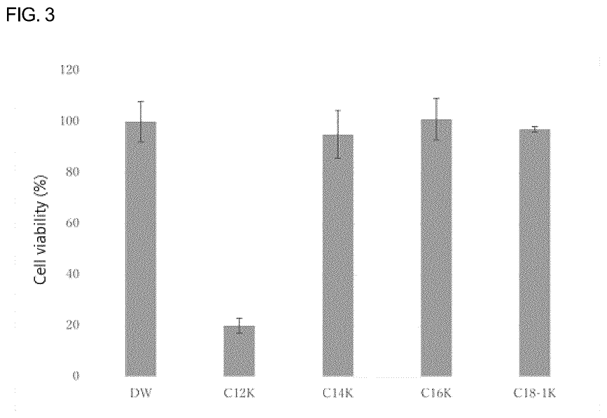

FIG. 3 shows the results. Based on OECD TG439, a case in which the cell viability is ≤50% was considered irritating, and a case in which the cell viability is >50% was considered non-irritating.

As shown in FIG. 3, the lauric acid salt, which is a salt of a C₁₂ saturated fatty acid, was determined to be irritating, and the salts of the saturated or unsaturated fatty acids having 14 or more carbon atoms were determined to be non-irritating.

### INDUSTRIAL APPLICABILITY

The cleaning agent composition of the present invention has good stability, is less irritating to the skin, and imparts an excellent feel during use. Thus, the cleaning agent composition of the present invention can be used for washing the human body (e.g., hair and body, including hands and face), dishes, houses, clothes, and anything in daily life.

1. A liquid cleaning agent composition comprising:
(a) fatty acid salts; and
(b) a sophorose lipid,
wherein
the fatty acid salts (a) comprise (a-1) at least one member selected from the group consisting of salts of C₁₄-C₂₂ saturated fatty acids and (a-2) at least one member selected from the group consisting of salts of C₁₄-C₂₂ unsaturated fatty acids, and
the components are contained in the following proportions:
A) the total amount of the component (a) is 2 to 35 mass % based on the cleaning agent composition taken as 100 mass %;
B) the proportion of the component (a-2) is 20 parts by mass or more based on 100 parts by mass of the total amount of the component (a); and
C) the proportion of the component (b) is 3 parts by mass or more per 100 parts by mass of the component (a-2).

2. The cleaning agent composition according to claim 1, wherein B) the proportion of the component (a-2) is 20 to 95 parts by mass based on 100 parts by mass of the total amount of the component (a).

3. The cleaning agent composition according to claim 2, wherein C) the proportion of the component (b) is 3 to 500 parts by mass per 100 parts by mass of the component (a-2).

4. The cleaning agent composition according to claim 2, wherein A) the total amount of the component (a) is 3 to 32 mass % based on the cleaning agent composition taken as 100 mass %.

5. The cleaning agent composition according to claim 2,
wherein
the component (a-1) is a salt of at least one member selected from the group consisting of myristic acid, palmitic acid, isopalmitic acid, stearic acid, isostearic acid, and arachidic acid, and
the component (a-2) is a salt of at least one member selected from the group consisting of oleic acid, linoleic acid, linolenic acid, palmitoleic acid, arachidonic acid, eicosenoic acid, erucic acid, eicosadienoic acid, docosadienoic acid, and mead acid.

6. A method for suppressing thermal deterioration of a cleaning agent composition comprising (a) fatty acid salts, the fatty acid salts (a) comprising (a-1) at least one member selected from the group consisting of salts of C₁₄-C₂₂ saturated fatty acids and (a-2) at least one member selected from the group consisting of salts of C₁₄-C₂₂ unsaturated fatty acids, the method comprising incorporating (b) a sophorose lipid in an amount of 3 parts by mass or more per 100 parts by mass of the component (a-2) in the cleaning agent composition.

7. The method according to claim 6, wherein the cleaning agent composition comprises a salt of at least one unsaturated fatty acid selected from the group consisting of linoleic acid, linolenic acid, eicosadienoic acid, docosadienoic acid, mead acid, and arachidonic acid as the component (a-2).

8. The method according to claim 7, wherein the cleaning agent composition comprises the component (a) and the component (a-2) in the following proportions:
A) the total amount of the component (a) is 2 to 35 mass % based on the cleaning agent composition taken as 100 mass %; and
B) the proportion of the component (a-2) is 20 parts by mass or more based on 100 parts by mass of the total amount of the component (a).

9. The method according to claim 8, wherein A) the total amount of the component (a) is 3 to 32 mass % based on the cleaning agent composition taken as 100 mass %.

10. The method according to claim 8, wherein B) the proportion of the component (a-2) is 20 to 95 parts by mass based on 100 parts by mass of the total amount of the component (a).

11. The method according to claim 7, wherein the proportion of the component (b) is 3 to 500 parts by mass per 100 parts by mass of the component (a-2).

12. The cleaning agent composition according to claim 1, wherein C) the proportion of the component (b) is 3 to 500 parts by mass per 100 parts by mass of the component (a-2).

13. The cleaning agent composition according to claim 1, wherein A) the total amount of the component (a) is 3 to 32 mass % based on the cleaning agent composition taken as 100 mass %.

14. The cleaning agent composition according to claim 1,
wherein
the component (a-1) is a salt of at least one member selected from the group consisting of myristic acid, palmitic acid, isopalmitic acid, stearic acid, isostearic acid, and arachidic acid, and
the component (a-2) is a salt of at least one member selected from the group consisting of oleic acid, linoleic acid, linolenic acid, palmitoleic acid, arachidonic acid, eicosenoic acid, erucic acid, eicosadienoic acid, docosadienoic acid, and mead acid.

15. The method according to claim 6, wherein the cleaning agent composition comprises the component (a) and the component (a-2) in the following proportions:
A) the total amount of the component (a) is 2 to 35 mass % based on the cleaning agent composition taken as 100 mass %; and
B) the proportion of the component (a-2) is 20 parts by mass or more based on 100 parts by mass of the total amount of the component (a).

16. The method according to claim 15, wherein A) the total amount of the component (a) is 3 to 32 mass % based on the cleaning agent composition taken as 100 mass %.

17. The method according to claim 15, wherein B) the proportion of the component (a-2) is 20 to 95 parts by mass based on 100 parts by mass of the total amount of the component (a).

18. The method according to claim 6, wherein the proportion of the component (b) is 3 to 500 parts by mass per 100 parts by mass of the component (a-2).
